(19) United States
(12) Patent Application Publication
(10) Pub. No.: US 2023/0181474 A1
(43) Pub. Date: Jun. 15, 2023

(54) TABLET COMPRISING OPICAPONE

(71) Applicants: Ono Pharmaceutical Co., Ltd., Osaka (JP); BIAL - PORTELA & CA, S.A., São Mamede Do Coronado (PT)

(72) Inventors: Koji Taniguchi, Osaka (JP); Noboru Sekiya, Osaka (JP); Teófilo Cardoso de Vasconcelos, São Mamede do Coronado (PT); Ricardo Jorge dos Santos Lima, São Mamede do Coronado (PT)

(21) Appl. No.: 17/925,460

(22) PCT Filed: May 26, 2021
(86) PCT No.: PCT/IB2021/054574

§ 371 Date: Nov. 15, 2022

(30) Foreign Application Priority Data
May 26, 2020 (JP) 2020-091745

(51) Int. Cl. A61K 9/20; A61K 31/4439

(52) U.S. Cl. CPC A61K 9/2013; A61K 9/2095; A61K 31/4439

(57) ABSTRACT

The present invention provides a tablet comprising opicapone or a pharmaceutically acceptable salt thereof, having reduced tableting faults during manufacture. It further relates to a tablet comprising opicapone or a pharmaceutically acceptable salt thereof, wherein the tablet comprises particles of opicapone or pharmaceutically acceptable salt thereof having the following particle size, and a manufacturing method thereof:(i) a maximum length D50 value of 45 µm or less, and/or (ii) a maximum length D90 value of 110 µm or less

### TECHNICAL FIELD

The present invention relates to a tablet comprising opicapone.

### BACKGROUND ART

Opicapone is a compound represented by the following formula (1):

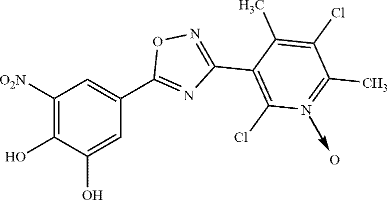 [SMILES: Cc1nc(Cl)c(-c2noc(-c3cc(O)c(O)c([N+](=O)[O-])c3)n2)c(C)c1Cl ; O]

That is, opicapone is 2,5-dichloro-3-[5-(3,4-dihydroxy-5-nitrophenyl)-1,2,4-oxadiazol-3-yl]-4,6-dimethylpyridine N-oxide. Note that 2,5-dichloro-3-[5-(3,4-dihydroxy-5-nitrophenyl)-1,2,4-oxadiazol-3-yl]-4,6-dimethylpyridine N-oxide may also be named 5-[3-(2,5-dichloro-4,6-dimethyl-1-oxy-pyridine-3-yl)-[1,2,4]oxadiazol-5-yl)-3-nitrobenzene-1,2-diol N-oxide.

Opicapone is a long-acting peripheral catechol-O-methyltransferase (COMT) inhibitor, and under the combined use of levodopa and a dopa-decarboxylase inhibitor, the activity of COMT, which is an alternative metabolic pathway of levodopa, is suppressed, and the duration of the effect of levodopa is extended, thereby improving diurnal fluctuation or end of dose fluctuation (wearing-off phenomenon) of symptoms in Parkinson’s disease.

In general, tableting faults may occur during the manufacture of tablets. The particle size of the drug substance is an important factor in tableting faults, and it is generally considered that the smaller the particle size, the larger the surface area of the particles, leading to the likelihood of tableting faults occurring (Journal of Pharmaceutical Sciences, Volume 107, pp. 2267 to 2282, 2018).

### SUMMARY OF INVENTION

The inventors of the present invention have recognized for the first time that when tablets comprising opicapone are manufactured, tableting faults occur. Accordingly, an object of the present invention is to provide a tablet having reduced tableting faults during manufacture.

The inventors of the present invention performed diligent studies to solve the problem. As a result, it was found that when manufacturing tablets comprising opicapone or a salt thereof as an active ingredient, a non-micronized drug substance is more prone to a tableting fault (sticking) compared with a micronized drug substance. This contradicts the general tendency that micronized drug substances are more likely to cause tableting faults since they have a larger specific surface area than non-micronized drug substances. Therefore, when drug substance profiles subjected to tableting were searched, it was ascertained that the maximum length of drug substance particles has an effect on tableting faults. In other words, it was discovered that by setting the maximum length of drug substance particles to a fixed level or less, tableting faults during tablet manufacture can be significantly reduced. Moreover, the inventors of the present invention discovered that tableting faults can be further reduced by increasing the amount of a lubricant added in the tablet by a fixed amount or more.

The present invention includes the following aspects.
A tablet comprising opicapone or a pharmaceutically acceptable salt thereof, wherein said opicapone or a pharmaceutically acceptable salt thereof is in the form of particles having the following particle size:
   (i) a maximum length D50 value of 45 µm or less, and/or
   (ii) a maximum length D90 value of 110 µm or less,
[1-1] the tablet according to , wherein the maximum length of the particle is a maximum length when measured using an iSPect DIA-10 (Shimadzu Corporation) and using a 10% polysorbate 20 aqueous solution as a dispersion medium,
[1-2] the tablet according to or [1-1], wherein depressions or roughness on the tablet surface is reduced (suppressed) in comparison to a tablet comprising particles of opicapone or a pharmaceutically acceptable salt thereof not having the particle size described,
the tablet according to any one of [1], [1-1], or [1-2], further comprising a lubricant in the tablet,
the tablet according to [2], wherein the lubricant comprises magnesium stearate,
the tablet according to [3], wherein the amount of magnesium stearate in the tablet is higher than 0.3% by mass,
the tablet according to any one of [1] to [4], wherein the particles are a micronized product (preferably a jet mill micronized product) of opicapone or a pharmaceutically acceptable salt thereof,
the tablet according to any one of [1] to [5], comprising a granulated product (preferably a granulated product obtained by a wet granulation method) comprising opicapone or a pharmaceutically acceptable salt thereof,
the tablet according to [6], wherein the granulated product comprising particles of opicapone or a pharmaceutically acceptable salt thereof, further comprises at least one diluent (preferably lactose hydrate), and/or at least one binder (preferably partially pregelatinised starch), and/or at least one disintegrant (preferably sodium starch glycolate),
the tablet according to [6] or [7], wherein the tablet obtained by tableting the granulated product is a mixture of the granulated product, at least one diluent (preferably lactose hydrate), at least one binder (preferably partially pregelatinised starch), and at least one lubricant (preferably magnesium stearate),
a method of manufacturing a tablet comprising opicapone or a pharmaceutically acceptable salt thereof, wherein the method of manufacturing the tablet includes a step of tableting particles of opicapone or a pharmaceutically acceptable salt thereof having the following particle size:
   (i) a maximum length D50 value of 45 µm or less, and/or
   (ii) a maximum length D90 value of 110 µm or less,
the manufacturing method according to [9], wherein the particles are a micronized product (preferably a jet mill micronized product) of opicapone or a pharmaceutically acceptable salt thereof,
the manufacturing method according to [9] or [10], further comprising a step of micronizing (preferably jet mill micronizing) opicapone or a pharmaceutically acceptable salt thereof to obtain particles of opicapone or a pharmaceutically acceptable salt thereof, having the specified maximum length.
the manufacturing method according to any one of [9] to [11], further comprising a step of granulating (preferably wet granulating) the particles of opicapone or a pharmaceutically acceptable salt thereof to obtain a granulated product, a step of mixing the granulated product with a lubricant, and a step of tableting the mixture.
a method of manufacturing a tablet comprising opicapone or a pharmaceutically acceptable salt thereof, wherein the method includes :
   (1) micronising opicapone or a pharmaceutically acceptable salt thereof to obtain particles of opicapone or a pharmaceutically acceptable salt thereof wherein the particle size thereof is:
      (i) a maximum length D50 value of 45 µm or less, and/or
      (ii) a maximum length D90 value of 110 µm or less;
   (2) granulating the particles obtained from step (1) to obtain a granulated product;
   (3) optionally, mixing the granulated product obtained in step (2) with a lubricant to obtain a mixture; and
   (4) tableting the granulated product obtained in step (2) or the mixture obtained in step (3),
the manufacturing method according to [13], wherein step (4) is a step of tableting the mixture obtained in step (3), and wherein the lubricant comprises magnesium stearate,
the manufacturing method according to [14], wherein the amount of magnesium stearate in the tablet is higher than 0.3% by mass, based on the total mass of the tablet.
the manufacturing method according to any one of [9] to [15], wherein an adhered amount of opicapone or a pharmaceutically acceptable salt thereof (drug substance) per 1 cm² of a surface of a tableting machine punch to which tablets contact is 1.0 µg/cm² or less after 450 tablets have been tableted,
a method of reducing (suppressing) tableting faults during manufacture of a tablet comprising opicapone or a pharmaceutically acceptable salt thereof, wherein the method includes using particles of opicapone or a pharmaceutically acceptable salt thereof having the particle size shown below:
   (A) a maximum length D50 value of 45 µm or less, and/or
   (B) a maximum length D90 value of 110 µm or less, and
the method according to [17], wherein tableting faults are depressions or roughness on a tablet surface.

### Effect of Invention

According to the method of manufacturing a tablet comprising opicapone or a pharmaceutically acceptable salt thereof of the present invention, it is possible to suppress or reduce tableting faults - that is, depressions or roughness on a tablet surface caused by a phenomenon wherein a tableting powder or drug substance adheres to a tableting machine punch (sticking). As a result, it is possible to manufacture high-quality tablets.

### BRIEF DESCRIPTION OF DRAWINGS

FIG. 1 illustrates the state of a tableting powder contact surface of a tableting machine punch and an adhered amount of a drug substance (µg) after approximately 450 tablets had been tableted of a preparation using a non-micronized and micronized opicapone drug substances shown in Table 1.

FIG. 2 illustrates the adhered amount of the drug substance (µg/cm²) per 1 cm² of a surface of a tableting machine punch to which the tablets contact after approximately 450 tablets had been tableted of the preparation using the non-micronized and micronized opicapone drug substances shown in Table 1.

### EMBODIMENTS OF THE INVENTION

A tablet of the present invention (hereinafter simply referred to as “tablet”) comprises opicapone or a pharmaceutically acceptable salt thereof.

Opicapone is a compound represented by the formula (1). Examples of a pharmaceutically acceptable salts thereof include an alkaline metal salt or an alkaline earth metal salt, and preferable examples include salts of strong base organic compounds such as sodium salts, potassium salts, or guanidine.

In the present specification, the lower limits and upper limits of all numerical ranges may be combined arbitrarily. Furthermore, each numerical value may include those rounded to a relevant value. For example, when there are two significant digits, the third digit is rounded off.

In the present specification, “maximum length” refers to a maximum length between two points on a contour of a particle, and this may be measured by the dynamic image analysis method. An example of the equipment used for measurement is the dynamic particle image analysis system iSpect DIA-10 (Shimadzu Corporation). In the present invention, the maximum length of particles of opicapone or a pharmaceutically acceptable salt thereof in terms of D50 value is less than 46 µm, preferably 45 µm or less, more preferably 40 µm or less, even more preferably 35 µm or less, and particularly preferably 30 µm or less or 29 µm or less. In the present invention, the maximum length of particles of opicapone or a pharmaceutically acceptable salt thereof in terms of D90 value is, for example, less than 111 µm, preferably 110 µm or less, more preferably 100 µm or less, even more preferably 90 µm or less, and particularly preferably 80 µm or less.

Furthermore, in the present invention, the lower limit of the maximum length of particles of opicapone or a pharmaceutically acceptable salt thereof is not particularly limited, but in terms of normal D50 value, it is 5 µm or more, 10 µm or more, 15 µm or more, 20 µm or more, 25 µm or more, preferably 10 µm or more, more preferably 15 µm or more, even more preferably 20 µm or more, and particularly preferably 25 µm or more. In the present invention, the lower limit of the maximum length of particles of opicapone or a pharmaceutically acceptable salt thereof is not particularly limited, but in terms of normal D90 value, it is 10 µm or more, 15 µm or more, 20 µm or more, 25 µm or more, 30 µm or more, 35 µm or more, 40 µm or more, 45 µm or more, 50 µm or more, 55 µm or more, 60 µm or more, preferably 45 µm or more, more preferably 50 µm or more, even more preferably 55 µm or more, and particularly preferably 60 µm or more.

Moreover, the D50 value and D90 value of the maximum length of particles of opicapone or a pharmaceutically acceptable salt thereof may be an arbitrary combination of those described above. The following (A) to (E) are given as examples of arbitrary combinations.
(A) D50 value: 45 µm or less (preferably 5 to 45 µm), D90 value: 110 µm or less (preferably 10 to 110 µm)
(B) D50 value: 40 µm or less (preferably 5 to 40 µm), D90 value: 100 µm or less (preferably 10 to 100 µm)
(C) D50 value: 35 µm or less (preferably 5 to 35 µm), D90 value: 90 µm or less (preferably 10 to 90 µm)
(D) D50 value: 30 µm or less (preferably 5 to 30 µm), D90 value: 80 µm or less (preferably 10 to 80 µm)
(E) D50 value: 29 µm or less (preferably 5 to 29 µm), D90 value: 80 µm or less (preferably 10 to 80 µm)

In the present specification, “equivalent circle diameter” refers to the diameter of a circle that has the same area as the projected area of a particle image, and this may be measured by the dynamic image analysis method. An example of the equipment used for measurement is the dynamic particle image analysis system iSpect DIA-10 (Shimadzu Corporation).

In the present specification, “micronized opicapone or a pharmaceutically acceptable salt thereof” (hereinafter also written as “micronized product”) may be opicapone or a pharmaceutically acceptable salt thereof micronized alone, and may be a micronized mixture of opicapone or a pharmaceutically acceptable salt thereof and a lubricant and/or other pharmaceutically acceptable additive.

In the present invention, examples of a “lubricant” include a fatty acid or a salt thereof (for example, stearic acid, stearate (for example, magnesium stearate (including light grade), calcium stearate, aluminium stearate), oleate (for example, sodium oleate)), talc, carnauba wax, white beeswax, beeswax, sucrose fatty acid ester, and lauryl sulfate (for example, sodium lauryl sulfate). In the present invention, stearate (for example, magnesium stearate, calcium stearate, and aluminium stearate) or stearic acid is preferable as the lubricant, stearate is more preferable, and magnesium stearate is most preferable. In the present invention, the lubricant may be included in the tablet powder together with other additives, or may be added by an external lubrication method in the tableting step.

In the present invention, the amount of the lubricant in the tablet (or in an uncoated tablet) is, for example, more than 0.3% by mass based on the total mass of the tablet, and is preferably 0.4% by mass or more, more preferably 0.5% by mass or more, and particularly preferably 1.0% by mass or more, based on the total mass of the tablet. The upper limit of the amount of lubricant is not particularly limited, provided that it does not affect the dissolution behaviour of the preparation or the hardness of the tablet, but it is, for example, less than 5.0% by mass, preferably 4.0% by mass or less, more preferably 3.0% by mass or less, and particularly preferably 2.0% by mass or less.

Furthermore, in the present invention, a suitable amount of the lubricant is preferably more than 2.3 parts by mass with respect to 100 parts by mass of opicapone or a pharmaceutically acceptable salt thereof, preferably 3.9 parts by mass or more, and more preferably 7.8 parts by mass or more. The upper limit is, for example, 40 parts by mass or less with respect to 100 parts by mass of opicapone or a pharmaceutically acceptable salt thereof, preferably 25 parts by mass or less.

The tablet of the present invention may comprise another pharmaceutically acceptable additive, including, for example, a diluent, a disintegrant, a disintegrant aid, a binder, a fluidizing agent, a flavouring agent, a deodorizer a surfactant, a fragrance, a colorant, an antioxidant, and a wetting agent. These may be used alone or two or more may be used in combination.

Examples of a diluent include lactose hydrate, anhydrous lactose, sucrose, purified sucrose, D-mannitol, D-sorbitol, glucose, trehalose, fructose, starch (for example, wheat starch, rice starch, corn starch, and potato starch), dextrin, macrogol 20000, light anhydrous silicic acid, calcium hydrogen phosphate hydrate, precipitated calcium carbonate, and kaolin.

Examples of a disintegrant include starch (for example, wheat starch, rice starch, corn starch, and potato starch), agar powder, sodium starch glycolate, partially pregelatinised starch, and D-mannitol.

Examples of a disintegrant aid include lactose, macrogol 1500, and macrogol 4000.

Examples of a binder include starch, partially pregelatinised starch, dextrin, pullulan, gum arabic, gelatine, tragacanth, sodium alginate, and polyvinylpyrrolidone.

Examples of a fluidizing agent include light anhydrous silicic acid, talc, and hydrous silicon dioxide.

Examples of a flavouring agent include sucrose, citric acid, ascorbic acid, tartaric acid, malic acid, aspartame, acesulfame potassium, thaumatin, sodium saccharin, dipotassium glycyrrhizinate, monosodium glutamate, sodium 5-inosinate, and sodium 5-guanylate.

Examples of a deodorizer include trehalose, malic acid, maltose, potassium gluconate, anise essential oil, vanilla essential oil, and cardamom essential oil.

Examples of a surfactant include polysorbate (such as polysorbate 80), polyoxyethylene-polyoxypropylene copolymer, and sodium lauryl sulfate.

Examples of a fragrance include lemon oil, orange oil, menthol, and peppermint oil.

Examples of a colorant include iron oxide (such as iron sesquioxide, yellow iron sesquioxide, black iron oxide) and titanium oxide.

Examples of an antioxidant include sodium ascorbate, L-cysteine, sodium sulfite, and vitamin E.

Examples of a wetting agent include polysorbate 80, sodium lauryl sulfate, sucrose fatty acid ester, macrogol, and hydroxypropyl cellulose.

An amount of a diluent (for example, lactose hydrate) in a tablet may be, based on the total mass of the tablet, for example, 60% by mass or more, 65% by mass or more, 70% by mass or more, 75% by mass or more, or 77% by mass or more, and may be 90% by mass or less, 85% by mass or less, or 83% by mass or less.

In one embodiment, a tablet comprises a granulated product and the amount of lactose hydrate as a diluent in the granulated product is, based on the total mass of a tablet, preferably 60 to 70% by mass, and more preferably 60 to 65% by mass, 65 to 70% by mass, or 62 to 67% by mass. Furthermore, in one embodiment, an amount of lactose hydrate as an diluent in a granulated product is, based on the total mass of a tablet, 60% by mass, 61% by mass, 62% by mass, 63% by mass, 64% by mass, 65% by mass, 66% by mass, 67% by mass, 68% by mass, 69% by mass, or 70% by mass.

In one embodiment, the amount of lactose hydrate as a diluent in a tablet is, based on the total mass of a tablet, preferably 70 to 85% by mass, more preferably 70 to 80% by mass or 75 to 85% by mass, and even more preferably 77 to 82% by mass. Furthermore, in one embodiment, the amount of lactose hydrate as a diluent in a tablet is, based on the total mass of the tablet, 70% by mass, 71% by mass, 72% by mass, 73% by mass, 74% by mass, 75% by mass, 76% by mass, 77% by mass, 78% by mass, 79% by mass, 80% by mass, 81% by mass, 82% by mass, 83% by mass, 84% by mass, or 85% by mass.

In one embodiment, a tablet is 100% by mass and comprises a granulated product, and the amount of extragranular lactose hydrate further added as a diluent to the granulated product is preferably 45 to 55% by mass, more preferably 45 to 50% by mass or 50 to 55% by mass, and even more preferably 45 to 48% by mass. Furthermore, in one embodiment, a tablet is 100% by mass and comprises a granulated product, and the amount of extragranular lactose hydrate as a diluent further added to a granulated product is 45% by mass, 46% by mass, 47% by mass, 48%by mass, 49% by mass, 50% by mass, 51% by mass, 52% by mass, 53% by mass, 54% by mass, or 55% by mass.

The amount of a binder (for example, partially pregelatinised starch) in the tablet may be, based on the total mass of the tablet, for example, 1% by mass or more, 2% by mass or more, 3% by mass or more, or 4% by mass or more, and may be 20% by mass or less or 15% by mass or less.

In one embodiment, the amount of partially pregelatinised starch as a binder in a granulated product is, based on the total mass of a tablet, preferably 1 to 10% by mass, more preferably 1 to 5% by mass, or 5 to 10% by mass, and even more preferably 5 to 10% by mass. Furthermore, in one embodiment, the amount of partially pregelatinised starch as a binder in a granulated product is, based on the total mass of a tablet, 1% by mass, 2% by mass, 3% by mass, 4% by mass, 5% by mass, 6% by mass, 7% by mass, 8% by mass, 9% by mass, or 10% by mass.

In one embodiment, the amount of partially pregelatinised starch as a binder in a tablet is, based on the total mass of the tablet, preferably 1 to 10% by mass, more preferably 1 to 5% by mass, 5 to 10% by mass, or 2 to 6% by mass, and even more preferably 2 to 6% by mass. Furthermore, in one embodiment, the amount of partially pregelatinised starch as a binder in a tablet is, based on the total mass of the tablet, 1% by mass, 2% by mass, 3% by mass, 4% by mass, 5% by mass, 6% by mass, 7% by mass, 8% by mass, 9% by mass, or 10% by mass.

The amount of a disintegrant (for example, sodium starch glycolate) in the tablet may be, based on the total mass of the tablet, for example, 1% by mass or more, 2% by mass or more, or 3% by mass or more, and may be 20% by mass or less or 15% by mass or less.

In one embodiment, the amount of sodium starch glycolate as a disintegrant in a granulated product is, based on the total mass of a tablet, preferably 1 to 10% by mass, more preferably 1 to 5% by mass, and even more preferably 1 to 2% by mass, 2 to 3% by mass, 3 to 4% by mass, or 4 to 5% by mass. Furthermore, in one embodiment, the amount of sodium starch glycolate as a disintegrant in a granulated product is, based on the total mass of a tablet, 1% by mass, 2% by mass, 3% by mass, 4% by mass, or 5% by mass.

In one embodiment, the amount of sodium starch glycolate as a disintegrant in a tablet is preferably 1 to 10% by mass, more preferably 1 to 5% by mass, and even more preferably 1 to 2% by mass, 2 to 3% by mass, 3 to 4% by mass, or 4 to 5% by mass. Furthermore, in one embodiment the amount of sodium starch glycolate in a tablet is, based on the total mass of the tablet, 1% by mass, 2% by mass, 3% by mass, 4% by mass, or 5% by mass.

In one embodiment, a tablet is 100% by mass and comprises a granulated product, and the amount of extragranular sodium starch glycolate further added as a disintegrant to a granulated product is preferably 0.5 to 5% by mass, and more preferably 0.5 to 2% by mass. Furthermore, in one embodiment, a tablet is 100% by mass and comprises a granulated product, and the amount of extragranular sodium starch glycolate further added as a disintegrant to a granulated product is 0.5% by mass, 1.0% by mass, 1.5% by mass, or 2.0% by mass.

One embodiment of the granulated product of the present invention is a granulated product comprising lactose hydrate as a diluent, partially pregelatinised starch as a binder, and sodium starch glycolate as a disintegrant.

One embodiment of the granulated product of the present invention is a granulated product comprising, based on the total mass of the granulated product, lactose hydrate (a) as a diluent at a content percentage of 60 to 65% by mass, partially pregelatinised starch (d) as a binder at a content percentage of 1 to 10% by mass, and sodium starch glycolate (e) as a disintegrant at a content percentage of 1 to 2% by mass.

One embodiment of the granulated product of the present invention is a granulated product comprising, based on the total mass of the granulated product, lactose hydrate (a) as an diluent at a content percentage of 60 to 65% by mass, partially pregelatinised starch (d) as a binder at a content percentage of 5 to 10% by mass, and sodium starch glycolate (e) as a disintegrant at a content percentage of 2 to 3% by mass.

One embodiment of the granulated product of the present invention is a granulated product comprising, based on the total mass of the granulated product, lactose hydrate (a) as a diluent at a content percentage of 65 to 70% by mass, partially pregelatinised starch (d) as a binder at a content percentage of 1 to 10% by mass, and sodium starch glycolate (e) as a disintegrant at a content percentage of 1 to 2% by mass.

One embodiment of the granulated product of the present invention is a granulated product comprising, based on the total mass of the granulated product, lactose hydrate (a) as a diluent at a content percentage of 65 to 70% by mass, partially pregelatinised starch (d) as a binder at a content percentage of 5 to 10% by mass, and sodium starch glycolate (e) as a disintegrant at a content percentage of 2 to 3% by mass.

One embodiment of the granulated product of the present invention is a granulated product comprising, based on the total mass of the granulated product, lactose hydrate (a) as a diluent at a content percentage of 62 to 67% by mass, partially pregelatinised starch (d) as a binder at a content percentage of 1 to 10% by mass, and sodium starch glycolate (e) as a disintegrant at a content percentage of 1 to 2% by mass.

One embodiment of the granulated product in the present invention is a granulated product comprising, based on the total mass of the granulated product, lactose hydrate (a) as a diluent at a content percentage of 62 to 77% by mass, partially pregelatinised starch (d) as a binder at a content percentage of 5 to 10% by mass, and sodium starch glycolate (e) as a disintegrant at a content percentage of 2 to 3% by mass.

One embodiment of the granulated product of the present invention is a granulated product comprising, based on the total mass of the granulated product, lactose hydrate (a) as a diluent at a content percentage of 62% by mass, partially pregelatinised starch (d) as a binder at a content percentage of 10% by mass, and sodium starch glycolate (e) as a disintegrant at a content percentage of 3% by mass.

One embodiment of the granulated product of the present invention is a granulated product comprising, based on the total mass of the granulated product, lactose hydrate (a) as a diluent at a content percentage of 63% by mass, partially pregelatinised starch (d) as a binder at a content percentage of 9 to 10% by mass, and sodium starch glycolate (e) as a disintegrant at a content percentage of 2 to 3% by mass (note that the total of (d) and (e) is 12% by mass).

One embodiment of the granulated product of the present invention is a granulated product comprising, based on the total mass of the granulated product, lactose hydrate (a) as a diluent at a content percentage of 64% by mass, partially pregelatinised starch (d) as a binder at a content percentage of 8 to 10% by mass, and sodium starch glycolate (e) as a disintegrant at a content percentage of 1 to 3% by mass (note that the total of (d) and (e) is 11% by mass).

One embodiment of the granulated product of the present invention is a granulated product comprising, based on the total mass of the granulated product, lactose hydrate (a) as a diluent at a content percentage of 65% by mass, partially pregelatinised starch (d) as a binder at a content percentage of 7 to 9% by mass, and sodium starch glycolate (e) as a disintegrant at a content percentage of 1 to 3% by mass (note that the total of (d) and (e) is 10% by mass).

One embodiment of the granulated product of the present invention is a granulated product comprising based on the total mass of the granulated product, lactose hydrate (a) as a diluent at a content percentage of 66% by mass, partially pregelatinised starch (d) as a binder at a content percentage of 6 to 8% by mass, and sodium starch glycolate (e) as a disintegrant at a content percentage of 1 to 3% by mass (note that the total of (d) and (e) is 9% by mass).

One embodiment of the granulated product of the present invention is a granulated product comprising, based on the total mass of the granulated product, lactose hydrate (a) as a diluent at a content percentage of 67% by mass, partially pregelatinised starch (d) as a binder at a content percentage of 5 to 7% by mass, and sodium starch glycolate (e) as a disintegrant at a content percentage of 1 to 3% by mass (note that the total of (d) and (e) is 8% by mass).

One embodiment of the tablet of the present invention is a tablet comprising lactose hydrate as a diluent, partially pregelatinised starch as a binder, and sodium starch glycolate as a disintegrant.

One embodiment of the tablet of the present invention is a tablet comprising, based on the total mass of the tablet, lactose hydrate (c) as a diluent at a content percentage of 75 to 80% by mass, partially pregelatinised starch (d) as a binder at a content percentage of 2 to 6% by mass, and sodium starch glycolate (g) as a disintegrant at a content percentage of 2 to 3% by mass.

One embodiment of the tablet of the present invention is a tablet comprising, based on the total mass of the tablet, lactose hydrate (c) as a diluent at a content percentage of 77 to 82% by mass, partially pregelatinised starch (d) as a binder at a content percentage of 2 to 6% by mass, and sodium starch glycolate (g) as a disintegrant at a content percentage of 2 to 3% by mass.

One embodiment of the tablet of the present invention is a tablet comprising, based on the total mass of the tablet, lactose hydrate (c) as a diluent at a content percentage of 80 to 85% by mass, partially pregelatinised starch (d) as a binder at a content percentage of 2 to 6% by mass, and sodium starch glycolate (g) as a disintegrant at a content percentage of 2 to 3% by mass.

One embodiment of the tablet of the present invention is a tablet comprising, based on the total mass of the tablet, lactose hydrate (c) as a diluent at a content percentage of 77% by mass, partially pregelatinised starch (d) as a binder at a content percentage of 6% by mass, and sodium starch glycolate (g) as a disintegrant at a content percentage of 3% by mass.

One embodiment of the tablet of the present invention is a tablet comprising, based on the total mass of the tablet, lactose hydrate (c) as a diluent at a content percentage of 78% by mass, partially pregelatinised starch (d) as a binder at a content percentage of 5 to 6% by mass, and sodium starch glycolate (g) as a disintegrant at a content percentage of 2 to 3% by mass (note that the total of (d) and (g) is 8% by mass).

One embodiment of the tablet of the present invention is a tablet comprising, based on the total mass of the tablet, lactose hydrate (c) as a diluent at a content percentage of 79% by mass, partially pregelatinised starch (d) as a binder at a content percentage of 4 to 5% by mass, and sodium starch glycolate (g) as a disintegrant at a content percentage of 2 to 3% by mass (note that the total of (d) and (g) is 7% by mass).

One embodiment of the tablet of the present invention is a tablet comprising, based on the total mass of the tablet, lactose hydrate (c) asa diluent at a content percentage of 80% by mass, partially pregelatinised starch (d) as a binder at a content percentage of 3 to 4% by mass, and sodium starch glycolate (g) as a disintegrant at a content percentage of 2 to 3% by mass (note that the total of (d) and (g) is 6% by mass).

One embodiment of the tablet of the present invention is a tablet comprising, based on the total mass of the tablet, lactose hydrate (c) as a diluent at a content percentage of 81% by mass, partially pregelatinised starch (d) as a binder at a content percentage of 3% by mass, and sodium starch glycolate (g) as a disintegrant at a content percentage of 2% by mass.

One embodiment of the tablet of the present invention is a tablet comprising, based on the total mass of the tablet, lactose hydrate (c) as a diluent at a content percentage of 82% by mass, partially pregelatinised starch (d) as a binder at a content percentage of 2% by mass, and sodium starch glycolate (g) as a disintegrant at a content percentage of 2% by mass.

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 40 to 48.7% by mass (preferably 45 to 48% by mass) and extragranular sodium starch glycolate (f) as a disintegrant at a content percentage of 0.5 to 3% by mass (preferably 0.5 to 2% by mass).

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 45% by mass and extragranular sodium starch glycolate (f) as a disintegrant at a content percentage of 3% by mass.

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 46% by mass and extragranular sodium starch glycolate (f) as a disintegrant at a content percentage of 2% by mass.

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 47% by mass and extragranular sodium starch glycolate (f) as a disintegrant at a content percentage of 1% by mass.

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 48% by mass and extragranular sodium starch glycolate (f) as a disintegrant at a content percentage of 1% by mass.

One embodiment of the tablet of the present invention is a tablet that is 100% by mass and comprises a granulated product, wherein 50.8% by mass is the granulated product and the tablet further comprises extragranular lactose hydrate (b) as a diluent at a content percentage of 48% by mass and extragranular sodium starch glycolate (f) as a disintegrant at a content percentage of 0.5% by mass.

The following tablets, C1 to C4, are given as suitable examples of the tablet of the present invention.

(C1) A tablet C1, wherein in the tablet, the total amount of the tablet is 100% by mass, and the content percentage of magnesium stearate is 0.5% by mass or more (in particular, 1.0 to 2.0% by mass).

(C2) A tablet C2, wherein the tablet C1 further comprises lactose hydrate

(C3) A tablet C3, wherein the tablet C1 or C2 further comprises partially pregelatinised starch.

(C4) A tablet C4, wherein the tablet C1, C2, or C3 further comprises sodium starch glycolate.

In the present invention, opicapone or a pharmaceutically acceptable salt thereof comprised in the tablet may be micronized. The micronization method is not particularly limited, and examples include air flow type (jetmill micronization or the like), rotary impact type (pin mill micronization, hammer mill micronization, or the like), tumbler type (ball mill micronization or the like), and wet type (bead mill micronization or the like). Among these, jet mill micronization is preferable.

When opicapone or a pharmaceutically acceptable salt thereof is micronized, the micronization step may be prior to the tablet manufacturing step ( particularly prior to the granulation step) or during the tablet manufacturing step (particularly, during the step of mixing opicapone or a pharmaceutically acceptable salt thereof with a pharmaceutically acceptable additive). For example, after micronizing the opicapone or a pharmaceutically acceptable salt thereof to obtain particles (micronized product) having the above-described particle size, it is possible to subject the micronized product to the granulation step together with a pharmaceutically acceptable additive, and furthermore, after micronizing a mixture comprising opicapone or a pharmaceutically acceptable salt thereof and a pharmaceutically acceptable additive to obtain a mixture comprising particles (micronized product) of opicapone or a pharmaceutically acceptable salt thereof having the above-described particle size, it is possible to subject this to the granulation step.

In the present invention, the method of manufacturing the tablet is not particularly limited, and it may include a granulation step, and may further include a particle size regulation step (including wet type or dry type particle size regulation) or a mixing step. The granulation step may be either wet granulation or dry granulation, but wet granulation is preferable. Examples of the wet granulation method include a stirring granulation method, a spray granulation method, a fluidized bed granulation method, a rolling granulation method, and an extrusion granulation method; however, a stirring granulation method is preferable.

Specifically, particles of opicapone or a pharmaceutically acceptable salt thereof may be granulated, if necessary, together with the above-described pharmaceutically acceptable additives to obtain a granulated product. The components and content in the granulated product may be arbitrarily selected from the above-described aspects.

Next, the obtained granulated product may be mixed, if necessary, with the above-described pharmaceutically acceptable additives, lubricant, or the like, and tableted to manufacture tablets. The components and content in the tablet may be arbitrarily selected from among the above-described aspects. Uncoated tablets may be obtained by tableting using a tableting machine (for example, a rotary tableting machine, a single-shot tableting machine, or the like). Tableting pressure is usually 1 to 35 kN/cm², preferably 2 to 30 kN/cm², more preferably 3 to 25 kN/cm², even more preferably 4 to 20 kN/cm², and particularly preferably 5 to 20 kN/cm² or 10 to 20 kN/cm².

In the present specification, “tableting fault” means a phenomenon in which the tablet powder or a drug substance adheres to a tabletting machine punch (sticking), or a depression or roughness on a tablet surface is caused by this phenomenon.

Tablets may, for example, be uncoated or coated tablets (for example, sugar-coated tablets and film-coated tablets). Of these, coated tablets are preferable, and film-coated tablets are more preferable.

In the present specification, “reduce tableting faults” means, for example, creating a state wherein there is no adhesion of the tablet powder or drug substance to the tabletting machine punch, or wherein adhesion is mild (hazy), or alternatively, creating a state wherein the adhered amount of the drug substance per 1 cm² adhered to a surface contacting tablets of the upper and lower punch is 0.60 µg/cm² or less after tabletting 450 tablets. Furthermore, this refers to creating a state wherein depressions and roughness on the tablet surface are reduced (suppressed) (the tablet surface is made to be smooth) in comparison to tablets comprising particles of opicapone or a pharmaceutically acceptable salt thereof which do not have the above-described particle size.

Depressions and roughness on the tablet surface may be evaluated not only by visual confirmation but also by, for example, the rough texture state of the tablet surface can be determined using a surface roughness shape measuring machine (JP H8-20537 A). Examples of equipment used for evaluation include the Surfcom 575A (Tokyo Seimitsu), Surfcom C5 (Tokyo Seimitsu), Surfcom 1400G (Tokyo Seimitsu), Surfcom TOUCH35 (Tokyo Seimitsu), Surfcom TOUCH40 (Tokyo Seimitsu), Surfcom TOUCH45 (Tokyo Seimitsu), Surfcom TOUCH50 (Tokyo Seimitsu), and Surfcom TOUCH550 (Tokyo Seimitsu).

A film-coated tablet is a tablet wherein an uncoated tablet is coated using a film coating. Film coatings usually include a base.

The base is usually a water-soluble base, and specific examples include methyl cellulose, ethyl cellulose, hydroxypropyl cellulose, hydroxypropyl methyl cellulose, hydroxypropyl methyl cellulose phthalate, hydroxypropyl methyl cellulose acetate succinate, carboxymethyl cellulose, polyvinyl acetal diethylaminoacetate, polyvinyl alcohol, polyvinyl pyrrolidone, dextrin, pullulan, aminoalkyl methacrylate copolymer, (meth)acrylate copolymer, carboxyvinyl polymer, sucrose, mannitol, and gelatin. The base may be used alone or in combination of two or more. At least one selected from the group consisting of polyvinyl alcohol, hydroxypropyl cellulose, and hydroxypropyl methyl cellulose is preferable as the base.

In addition to the base, the film coating may comprise a pharmaceutically acceptable additive, such as a lubricant, plasticiser, or colourant. These may be used alone or in combination of two or more.

Examples of a lubricant include talc.

Examples of a plasticizer include polyethylene glycol, triacetin, medium-chain fatty acid triglyceride, acetylglycerol fatty acid ester, triethyl citrate, and combinations of two or more of these.

Examples of a colourant include iron oxide (iron sesquioxide, yellow iron sesquioxide, black iron oxide, or the like) titanium oxide, and combinations of two or more of these.

A mass ratio of the film coating is, for example, 1 to 15 parts by mass with respect to 100 parts by mass of an uncoated tablet, and preferably 2 to 10 parts by mass.

These tablets are useful in the treatment or prevention of Parkinson’s disease, and more preferably useful for the improvement of diurnal fluctuation or end of dose fluctuations (wearing-off phenomenon) of symptoms in Parkinson’s disease when used in combination with a levodopa-containing preparation.

The administration frequency of the tablets is not particularly limited, and may be, for example, once, twice, or three times a day, once every two days, or the like, and preferably once a day. The time of administration of the tablet may be in the morning, noon, evening, before bedtime, before or after administration of the other drug, or one hour or more before or after meals, preferably one hour or more before or after administration of other drugs or before or after meals.

In the present invention, the content of opicapone or a pharmaceutically acceptable salt thereof in the tablet is, for example, 20 to 100 mg, preferably 25 mg or 50 mg, and more preferably 25 mg.

The tablets may be combined with other drugs, such as drugs for enhancing action, drugs for reducing side effects, and drugs for preventing or treating other diseases (such as lifestyle-related diseases), or the like. Examples of other drugs combined with the tablets of the present invention include levodopa, dopa decarboxylase inhibitors (carbidopa, benserazide), dopamine agonists (bromocriptine, pergolide, talipexole, or the like), monoamine oxidase B inhibitors (selegiline, rasagiline,), amantadine, anticholinergic agents, droxidopa, zonisamide, or istradefylline, preferably levodopa or dopa decarboxylase inhibitors. These drugs may be administered in the form of a combination drug in which both components are combined in one preparation, or may be administered in the form of separate preparations. When administered as separate preparations, simultaneous administration and staggered administration are included. In addition, in terms of staggered administration, the compound of the present invention may be administered first and the other drug may be administered later, or the other drug may be administered first and the compound of the present invention may be administered later. The respective administration methods may be the same or different.

“Including”, “containing”, “comprising” or “having” used in the present specification encompasses, but is not limited to, the meanings of “consisting essentially of” and “consisting of”.

### EXAMPLES

Hereinafter, the present invention will be described in detail according to examples, but the present invention is not limited thereto. In the examples “%” means “% by mass”, unless otherwise specified.

### Example 1: Micronization of Opicapone Drug Substance

The opicapone drug substance was micronized under the following conditions to obtain micronized products A1 to A4.

### Micronization Conditions of Micronizedproducts A1 and A2

Micronization machine: MC Jet mill MC200 (Dietrich Engineering Consultants sa)
Supply speed: 200 to 400 g per 30 seconds
Micronisation pressure: 3.0 to 7.0 bar (300 to 700 kPa)

### Micronization Conditions of Micronizedproducts A3 and A4

Micronization machine: MC Jet mill MC200 (Dietrich Engineering Consultants sa)
Supply speed: 100 to 400 g per 30 seconds
Micronisation pressure: 3.0 to 4.0 bar (300 to 400 kPa)

### Example 2: Evaluation of Particle Size

The particle sizes of the non-micronized product and the micronized products A1 to A4 prepared in Example 1 using the opicapone drug substance were measured under the following conditions.

### Measurement Conditions

Measurement equipment: Dynamic particle image analysis system iSpect DIA-10 (Shimadzu Corporation)
Evaluation items: Equivalent circle diameter, maximum length
Dispersion medium: 10% polysorbate 20 aqueous solution
Sample: Approximately 10 mg of the measurement sample was placed in 1 ml of the dispersion medium, suspended, and then diluted 100-fold.

### Results and Observations

The particle size measurement results of the non-micronized product and micronized products A1 to A4 are shown in the following table. Although no considerable difference in equivalent circle diameter was observed between the non-micronized product and the micronized products A1 to A4, the non-micronized product exhibited a larger value for maximum length than the micronized products A1 to A4. Furthermore, it was found that the maximum lengths D50 and D90 have clearer differences in particle size between the non-micronized product and the micronized products than equivalent circle diameter.

TABLE 1

| | | Non-micronized Product | Micronized Product A 1 | Micronized Product A2 | Micronized Product A3 | Micronized Product A4 |
|---|---|---|---|---|---|---|
| Equivalent Circle Diameter [µm] | D 1 0 | 6 | 7 | 7 | 7 | 8 |
| | D 5 0 | 13 | 11 | 11 | 11 | 12 |
| | D 9 0 | 23 | 1 9 | 18 | 19 | 21 |
| Maximum Length [µm] | D 1 0 | 16 | 16 | 16 | 15 | 17 |
| | D 5 0 | 4 6 | 28 | 26 | 2 5 | 29 |
| | D 9 0 | 111 | 63 | 60 | 73 | 80 |

### Example 3: Preparation of Test Formulations

The formulation and loading of Formulation Examples B1 to B6 are shown in the tables below. The non-micronized and micronized drug substances shown in the table above were used for the respective formulation examples. Furthermore, the numbers in Table 2 indicate the added amount (% by mass) of the drug substance or additive when the entire uncoated tablet is 100% by mass, and the numbers in Table 2, Table 3, and Table 4 indicate the added amount (g) of the drug substance or additive. The formulation examples B5 and B6 use the same granulated product, and the amount of extragranular lactose hydrate (b) added to the granulated product is increased by 0.5 g in B6 compared to B5.

TABLE 2

<Formulation of Tablets> (% by mass)

| Formulation Example | B 1 | B2 | B3 | B 4 | B5 | B 6 |
|---|---|---|---|---|---|---|
| Drug Substance Used | Non-micronized Product | Micronized Product A1 | Micronized Product A2 | Micronized Product A3 | Micronized Product A4 | . Micronized Product A4 |
| Opicapone | 12.8 | 12.8 | 12.8 | 12.8 | 12.8 | 12.8 |
| Mixture of lactose hydrate (a), partially pregelatinised starch (d), and sodium starch glycolate (e) | 38.0 | 38.0 | 38.0 | 38.0 | 38.0 | 38.0 |
| Granulated product | 50.8 | 50.8 | 50.8 | 50.8 | 50.8 | 50.8 |
| Mixture of lactose hydrate (b) and sodium starch glycolate (f) | 48. 2 | 48.2 | 48.2 | 48.2 | 48.2 | 48.7 |
| Magnesium stearate | 1.0 | 1.0 | 1.0 | 1.0 | 1.0 | 0.5 |
| Total (Tableting powder) i | 100.0 | 100.0 | 100.0 | 100.0 | 100.0 | 100.0 |

TABLE 3

<Granulation> Granulation Load (g)

| Formulation Example | B 1 | B2 | B3 | B4 | B 5 ~ B 6 |
|---|---|---|---|---|---|
| Drug Substance Used | Non-micronized Product | Micronized Product A1 | Micronized Product A2 | Micronized Product A3 | Micronized Product A4 |
| Opicapone | 40 | 900 | 900 | 900 | 00 |
| Mixture of lactose hydrate, partially pregelatinised starch, and sodium starch glycolate | 118.4 | 2664 | 2664 | 2664 | 2664 |
| Total (Granulated product) | 158.4 | 256.4 | 3564 | 3564 | 3564 |

TABLE 4

<Tableting> Mixture Load (g)

| Formulation Example | B 1 | B 2 | B3 | B 4 | B 5 | B 6 |
|---|---|---|---|---|---|---|
| Granulated Product | 99 | 2772 | 2772 | 2772 | 2970 | 99 |
| Mixture of lactose hydrate and sodium starch glycolate | 94 | 2632 | 2632 | 263223 | 2820 | 95 |
| Magnesium stearate | 2 | 56 | 56 | 56 | 60 | 1 |
| Total (Tableting powder) | 195 | 5460 | 5460 | 5460 | 5850 | 195 |

The manufacturing method of formulation examples B1 to B6 is as follows.

Opicapone (non-micronized product,micronized products A1 to A4), lactose hydrate, partially pregelatinised starch, and sodium starch glycolate were placed in a high-speed stirring granulator FM-VG-25 or FM-VG-01 (manufactured by Powrex Corporation), purified water was added, and the mixture was granulated for seven minutes to obtain a granulated product.

Lactose hydrate, sodium starch glycolate, and magnesium stearate were mixed with the granulated product to obtain a tablet powder. A rotary-type tableting machine VEL2 (Kikusui Seisakusho, Ltd.) was used to tablet the tabletting powder at a pressure of approximately 10 kN so that the tablet weight was 195 mg, thereby obtaining tablets (oval type, major axis 11.5 mm, minor axis 5 mm). The surface area of the tableting machine punch used for tableting was 50.883 cm² per piece, and the surface area of the upper and lower punches was 101.766 cm².

### Example 4: Evaluation of Tableting Faults

### Evaluation Method

After preparing 450 respective tablets of the Formulation Examples B 1 to B6, photographs were taken of the tableting powder contact surface of the tableting machine punch used in the tableting step.

Furthermore, the adhesion state of the drug substance to the punch was visually confirmed, and when it was determined that the drug substance was attached, “x” was marked, when it was determined there was haze (adhesion of the drug substance was mild), “Δ” was marked, and when it was determined that the drug substance was not adhered, “o” was marked.

Moreover, by wiping the tableting powder contact surface of the upper and lower punches after tableting and quantifying the amount of drug substance, the total amount of drug substance adhered to the upper and lower punches (adhered amount of drug substance (µg)) was calculated. The adhered amount of drug substance per 1 cm² of the tableting machine punch (µg/cm²) was calculated from the calculated adhered amount of drug substance. Specifically, one swab (K-PINE sterilized, manufactured by Kawamoto Corporation) containing 2 ml of 0.5 w/v% citric acid aqueous solution and Japanese Pharmacopoeia absolute ethanol mixture (1:1) was held and wiped thoroughly 20 times or more so that the portion containing the solvent made contact with the tableting powder contact surface.

Next, another swab containing 1 ml of Japanese Pharmacopoeia absolute ethanol was held and wiped thoroughly 20 times or more so that the portion containing the solvent made contact with the wiping portion. Exactly 17 ml of a sample solvent (water/acetonitrile mixed solution = 1/1) was added to a container containing the two swabs that had been wiped, and this was shaken. Opicapone was extracted by pressing a swab against the inner wall surface of the container to extract the solvent.

The extract was appropriately diluted and the content of opicapone in the extract was measured by liquid chromatography. The measurement conditions were as follows. Note that an opicapone standard solution was adjusted by weighing an opicapone standard substance and adding a sample solvent so that the concentration of opicapone was 0.5 µg/ml.

### Test Conditions

Detector: Ultraviolet absorption spectrophotometer (measurement wavelength: 275 nm)
Column: XTerra RP18 (length 10 cm, inner diameter 4.6 mm, particle size 3.5 µm, Waters)
Column temperature: Constant temperature around 30° C.
Mobile phase: 10 mmol/1 of potassium dihydrogen phosphate solution (pH 2.2)/acetonitrile (11:9)
Flow rate: 1.0 ml/min
Sample cooler temperature: 10° C.
Analysis time: 12 minutes

### Calculation Method

Adhered amount of drug substance (µg) = Ms X P X (A_{T}/Aₛ) X (½R) X k
Ms: Weighed amount of opicapone standard substance (mg)
P: Purity correction coefficient of opicapone standard material
A_{T}: Peak area of opicapone
Aₛ: Mean value of peak area of opicapone
R: Recovery rate calculated by validation (%)
k: Dilution factor

### Results and Observations

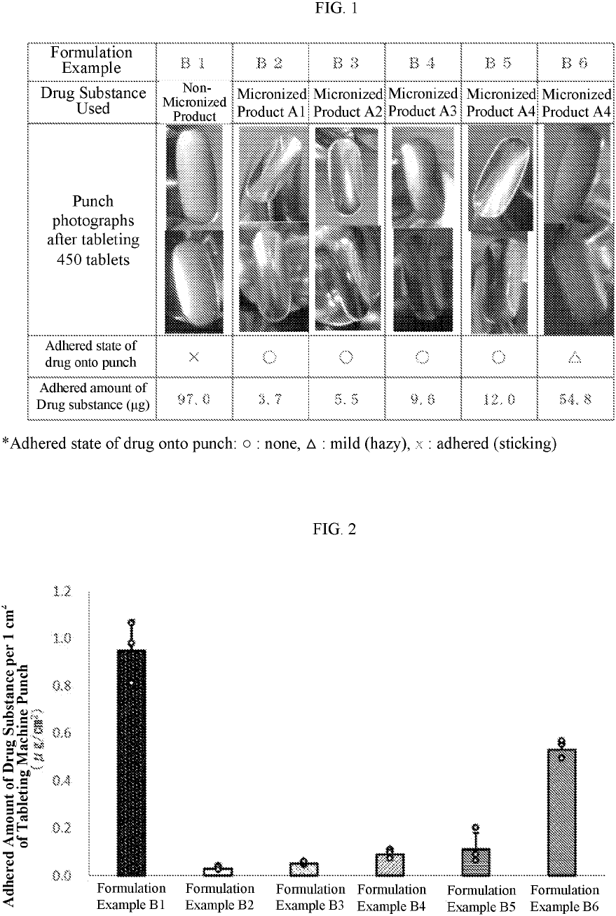

Regarding adhesion of the drug to the tableting machine punch after tableting approximately 450 tablets of the formulations manufactured using the non-micronized drug substance and micronized drug substances (formulation examples B1 to B5) and the formulations containing 1.0 to 0.5% of a lubricant (formulation examples B5 and B6), visual confirmation of the state of the drug adhered to the tableting machine punch and quantitative evaluation of the adhered amount of drug substance on the tableting powder contact surface of the tableting machine punch was carried out. FIG. 1 shows a photograph of the tableting powder contact surface of the tableting machine punch and illustrates the measurement results of the adhered amount of drug substance on the tableting machine punch, and FIG. 2 illustrates the measurement results of the adhered amount of drug substance per surface area of the tableting machine punch.

As a result of examining the effects when using the non-micronized drug substance and the micronized drug substances (Formulation Examples B1 to B5), in Formulation Example B1 using the non-micronized drug substance, the drug had strong adhesion to the tableting powder contact surface of the tableting machine punch after tableting, and the amount of drug substance adhered to the tableting machine punch was approximately 1.0 µg/cm². On the other hand, in Formulation Examples B2 to B5 using micronized drug substances, adhesion to the tableting powder contact surface of the tableting machine punch was not observed, and the adhered amount of drug substance to the tableting machine punch was as low as approximately 0.1 µg/cm² or less.

As a result of examining the effects of the amount of lubricant in the formulation having an amount of 0.5% of lubricant (Formulation Example B6), adhesion to the tableting powder contact surface of the tableting machine punch after tableting was mild, or hazy, and the adhered amount of drug substance to the tableting machine punch was approximately 0.5 µg/cm².

### INDUSTRIAL APPLICABILITY

Since tableting faults during manufacture are reduced, the present invention provides a hogh-quality tablet comprising opicapone or a pharmaceutically acceptable salt thereof.

1. A tablet comprising opicapone or a pharmaceutically acceptable salt thereof, wherein the tablet comprises particles of opicapone or a pharmaceutically acceptable salt thereof having the following particle size:
(i) a maximum length D50 value of 45 µm or less, and/or
(ii) a maximum length D90 value of 110 µm or less.

2. The tablet according to claim 1, further comprising a lubricant in the tablet.

3. The tablet according to claim 2, wherein the lubricant comprises magnesium stearate.

4. The tablet according to claim 3, wherein the amount of magnesium stearate in the tablet is higher than 0.3% by mass, based on the total mass of the tablet.

5. The tablet according to claim 1, wherein the particles of opicapone or a pharmaceutically acceptable salt thereof are a micronized product of opicapone or a pharmaceutically acceptable salt thereof.

6. The tablet according to any one of claim 1, comprising a granulated product comprising the particles of opicapone or a pharmaceutically acceptable salt thereof.

7. The tablet according to claim 6, wherein the granulated product comprises the particles of opicapone or a pharmaceutically acceptable salt thereof, at least one diluent, at least one binder, and at least one disintegrant.

8. The tablet according to claim 7, wherein the tablet obtained by tableting the granulated product is a mixture of the granulated product, at least one diluent, at least one binder, and at least one lubricant.

9. A method of manufacturing a tablet comprising opicapone or a pharmaceutically acceptable salt thereof according to claim 1, wherein the method includes a step of tableting particles of opicapone or a pharmaceutically acceptable salt thereof having the following particle size:
(i) a maximum length D50 value of 45 µm or less, and/or
(ii) a maximum length D90 value of 110 µm or less.

10. The manufacturing method according to claim 9, wherein the particles of opicapone or a pharmaceutically acceptable salt thereof are a micronized product of opicapone or a pharmaceutically acceptable salt thereof.

11. The manufacturing method according to claim 9, further characterised by having a step of micronizing opicapone or a pharmaceutically acceptable salt thereof to obtain the particles of opicapone or a pharmaceutically acceptable salt thereof.

12. The manufacturing method according to claim 9, further characterised by having a step of granulating the particles of opicapone or a pharmaceutically acceptable salt thereof to obtain a granulated product, a step of mixing the granulated product and a lubricant, and a step of tableting the resulting mixture.

13. A method of manufacturing a tablet comprising opicapone or a pharmaceutically acceptable salt thereof according to claim 1, wherein the method includes:
(1) micronizing opicapone or a pharmaceutically acceptable salt thereof to obtain particles of opicapone or a pharmaceutically acceptable salt thereof wherein the particle size thereof is:
(i) a maximum length D50 value of 45 µm or less, and/or
(ii) a maximum length D90 value of 110 µm or less;
(2) granulating the particles to obtain a granulated product;
(3) optionally, mixing the granulated product obtained in step (2) together with a lubricant to obtain a mixture; and
(4) tableting the granulated product obtained in step (2) or the mixture obtained in step (3).

14. The manufacturing method according to claim 13, wherein step (4) is a step of tableting the mixture obtained in step (3), and the lubricant comprises magnesium stearate.

15. The manufacturing method according to claim 14, wherein the amount of the magnesium stearate in the tablet is higher than 0.3% by mass, based on the total mass of the tablet.

16. The manufacturing method according to claim 9, wherein the adhered amount of opicapone or a pharmaceutically acceptable salt thereof per 1 cm² of a surface of a tableting machine punch to which tablets contact is 1.0 µg/cm² or less after 450 tablets have been tableted.

17. A method of reducing tableting faults during manufacture of a tablet comprising opicapone or a pharmaceutically acceptable salt thereof according to claim 1, wherein the method is characterised by using particles of opicapone or a pharmaceutically acceptable salt thereof having the particle size shown below:
(A) a maximum length D50 value of 45 µm or less, and/or
(B) a maximum length D90 value of 110 µm or less.

18. The method according to claim 17, wherein tableting faults are depressions or roughness on a tablet surface.

19. The tablet according to claim 4, wherein the particles of opicapone or a pharmaceutically acceptable salt thereof are a micronized product of opicapone or a pharmaceutically acceptable salt thereof.

20. The tablet according to claim 19, comprising a granulated product comprising the particles of opicapone or a pharmaceutically acceptable salt thereof.
